(19) United States
(12) Patent Application Publication
(10) Pub. No.: US 2018/0289248 A1
(43) Pub. Date: Oct. 11, 2018

(54) CLEANING STRUCTURE FOR ENDOSCOPE LENS

(71) Applicant: EVERICH BIOTECOLOGY CORPORATION, Zhubei City (TW)

(72) Inventor: Shih-Chu Chen, Zubei City (TW)

(21) Appl. No.: 15/481,440

(22) Filed: Apr. 6, 2017

(51) Int. Cl. A61B 1/12; A61B 1/00

(52) U.S. Cl. CPC A61B 1/12; A61B 1/00135

(57) ABSTRACT

A cleaning structure for endoscope lens includes a sleeve main body having a fitting hole, at least one first axial fluid passage and a first opening. The fitting hole communicates with two ends of the sleeve main body. The first axial fluid passage is axially formed in a wall of the sleeve main body. The first opening is formed at one end of the sleeve main body in communication with the first axial fluid passage. The sleeve main body is fitted around the lens of the endoscope. A fluid is supplied to the fluid passage structure of the sleeve main body and ejected from the opening to clean up the surface of the lens of the endoscope so as to keep the surface of the lens clean.

### BACKGROUND OF THE INVENTION

### 1. Field of the Invention

The present invention relates generally to a cleaning structure for endoscope lens, and more particularly to a cleaning structure for endoscope lens, which is able to clean up the surface of the lens of the endoscope.

### 2. Description of the Related Art

Currently, a conventional endoscope is widely used in a site where human eyes cannot directly inspect. The endoscope is widely used in both industrial field and medical field. In the industrial field, the endoscope is widely applied to a fine fissure or bent and narrow duct. In the medical field, the endoscope is often applied to minimally invasive surgery. The use of the endoscope is advantageous in that the wound of the minimally invasive surgery is minimized, the surgery time is shortened and the recovery of the wound is speeded.

However, in both the industrial field and medical field, the application of the endoscope has the same bother. That is, in working, in case the lens of the endoscope is contaminated, it is impossible to immediately clean up the lens. To solve this problem, a vehicle wiper-like cleaning device has been developed for cleaning the endoscope lens. However, when the endoscope is applied to a medical usage, the small-diameter endoscope entering human body is used to ablate or cut off the internal necrotic tissue. At this time, the internal blood and muscle tissue are apt to splash and attach to the surface of the lens of the endoscope. The wiper-like cleaning device is able to partially clean off the blood covering the lens. However, the wiper-like cleaning device can hardly completely clean off the muscle tissue attaching to the surface of the lens. In the case that the lens of the endoscope is covered by the dirt, it will be impossible to successfully perform the minimally invasive surgery. Under such circumstance, it is necessary to retreat the endoscope from the human body and then clean up the lens of the endoscope and then extend the endoscope into the human body again to proceed with the surgery. Such procedure is quite time-consuming and troublesome. For example, in the case that in cell sampling, the lens of the endoscope is contaminated and covered, it is necessary to retreat the endoscope from the human body and then clean up the lens of the endoscope and then extend the endoscope into the human body again to proceed with the cell sampling. Under such circumstance, it often costs much time to find out the sampled place again. This is quite inconvenient.

It is therefore tried by the applicant to provide a cleaning structure for endoscope lens, which is able to conveniently clean up the surface of the lens of the endoscope.

### SUMMARY OF THE INVENTION

It is therefore a primary object of the present invention to provide a cleaning structure for endoscope lens, which is able to conveniently clean up the lens of the endoscope.

To achieve the above and other objects, the cleaning structure for endoscope lens of the present invention includes a sleeve main body having a fitting hole, at least one first axial fluid passage and a first opening. The fitting hole communicates with two ends of the sleeve main body. The first axial fluid passage is axially formed in a wall of the sleeve main body. The first opening is formed at one end of the sleeve main body in communication with the first axial fluid passage.

The sleeve main body is fitted around the lens of the endoscope. A fluid is supplied to the fluid passage structure of the sleeve main body and ejected from the opening to immediately clean off the dirt attaching on the surface of the lens of the endoscope. The cleaning structure for endoscope lens of the present invention is able to conveniently clean up the lens of the endoscope to solve the shortcoming of the conventional wiper-like cleaning device that it is inconvenient to clean off the dirt attaching to the lens of the endoscope.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a perspective view of a first embodiment of the cleaning structure for endoscope lens of the present invention;

FIG. 2 is a sectional view of the first embodiment of the cleaning structure for endoscope lens of the present invention;

FIG. 3 is a sectional view of a second embodiment of the cleaning structure for endoscope lens of the present invention;

FIG. 4 is a sectional view of a third embodiment of the cleaning structure for endoscope lens of the present invention;

FIG. 5 is a sectional view of a fourth embodiment of the cleaning structure for endoscope lens of the present invention; and

FIG. 6 is a sectional view of a fifth embodiment of the cleaning structure for endoscope lens of the present invention.

### DETAILED DESCRIPTION OF THE PREFERRED EMBODIMENTS

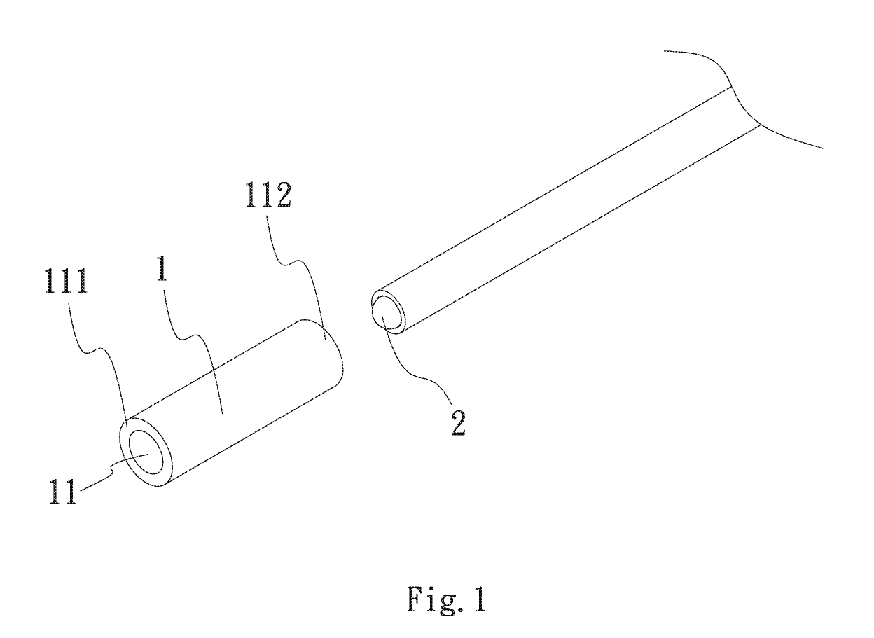

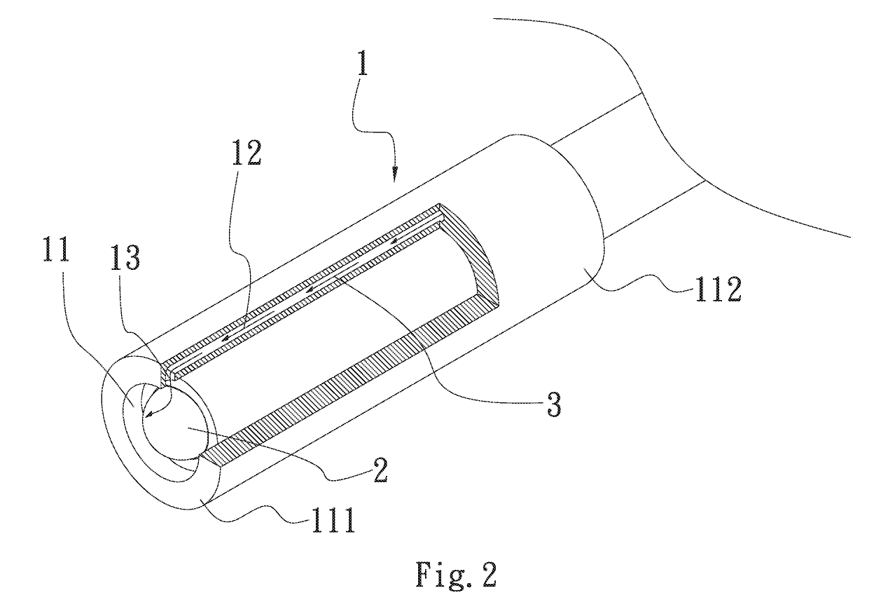

Please refer to FIGS. 1 and 2. FIG. 1 is a perspective view of a first embodiment of the cleaning structure for endoscope lens of the present invention. FIG. 2 is a sectional view of the first embodiment of the cleaning structure for endoscope lens of the present invention. According to the first embodiment, the cleaning structure for endoscope lens of the present invention includes a sleeve main body 1 having a fitting hole 11, at least one first axial fluid passage 12 and a first opening 13. The fitting hole 11 communicates with two ends of the sleeve main body 1. The first axial fluid passage 12 is axially formed in a wall of the sleeve main body 1. The first opening 13 is formed at one end of the sleeve main body 1 in communication with the first axial fluid passage 12.

The sleeve main body 1 has a first end 111 and a second end 112 respectively positioned at two ends of the sleeve main body 1. The first opening 13 is selectively disposed at one of the first and second ends 111,112. In this embodiment, the first opening 13 is, but not limited to, disposed at the first end 111. The sleeve main body 1 is made of a material selected from a group consisting of rubber, silicone, plastic, metal and glass.

In the cleaning structure for endoscope lens of the present invention, the fitting hole 11 of the sleeve main body 1 is fitted around the lens 2 of an endoscope. The lens 2 is positioned in a position where the first end 111 of the sleeve main body 1 is positioned. The cleaning structure for endoscope lens of the present invention provides a fluid 3 to flow through the first axial fluid passage 12 toward the first opening 13 and eject from the first opening 13. The first opening 13 is radially directed to the lens 2 of the endoscope so that the fluid 3 can clean off the dirt on the surface of the lens 2 of the endoscope. The fluid 3 can be a vapor fluid or a liquid fluid.

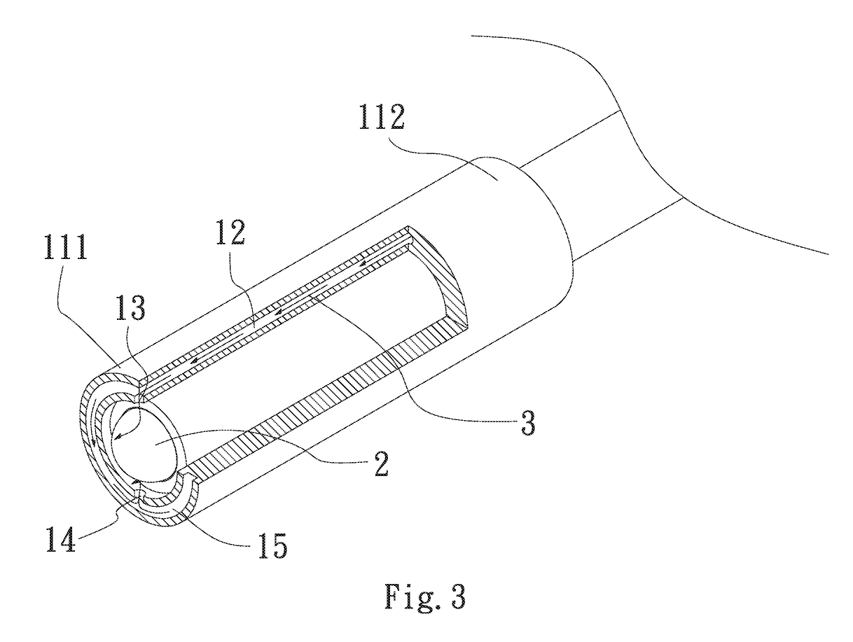

Please now refer to FIG. 3, which is a sectional view of a second embodiment of the cleaning structure for endoscope lens of the present invention. The second embodiment is partially identical to the first embodiment in structure and thus will not be repeatedly described hereinafter. The second embodiment is different from the first embodiment in that the sleeve main body 1 further has a second opening 14 and a circumferential fluid passage 15. The second opening 14 is selectively disposed at one of the first and second ends 111,112. The second opening 14 is in communication with the first axial fluid passage 12. The fluid 3 can flow through the first axial fluid passage 12 to the circumferential fluid passage 15 and then eject from the second opening 14. The second opening 14 is radially directed to the lens 2 of the endoscope so that the fluid 3 can directly clean off the dirt on the surface of the lens 2 of the endoscope as in the first embodiment.

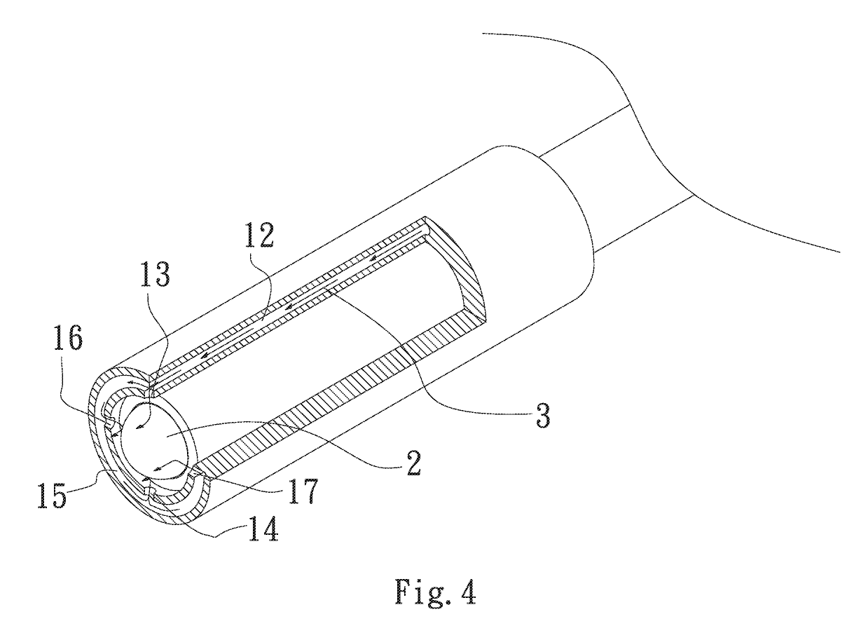

Please now refer to FIG. 4, which is a sectional view of a third embodiment of the cleaning structure for endoscope lens of the present invention. The third embodiment is partially identical to the second embodiment in structure and thus will not be repeatedly described hereinafter. The third embodiment is different from the second embodiment in that the sleeve main body 1 further has a third opening 16, a fourth opening 17. The third and fourth openings 16, 17 communicate with the first axial fluid passage 12, the first and second openings 13, 14 and the circumferential fluid passage 15. The fluid 3 can flow through the first axial fluid passage 12 to the circumferential fluid passage 15 and then eject from the first, second, third and fourth openings 13, 14, 16 and 17. The first, second, third and fourth openings 13, 14, 16 and 17 and the circumferential fluid passage 15 are radially directed to the lens 2 of the endoscope so that the fluid 3 can directly clean off the dirt on the surface of the lens 2 of the endoscope.

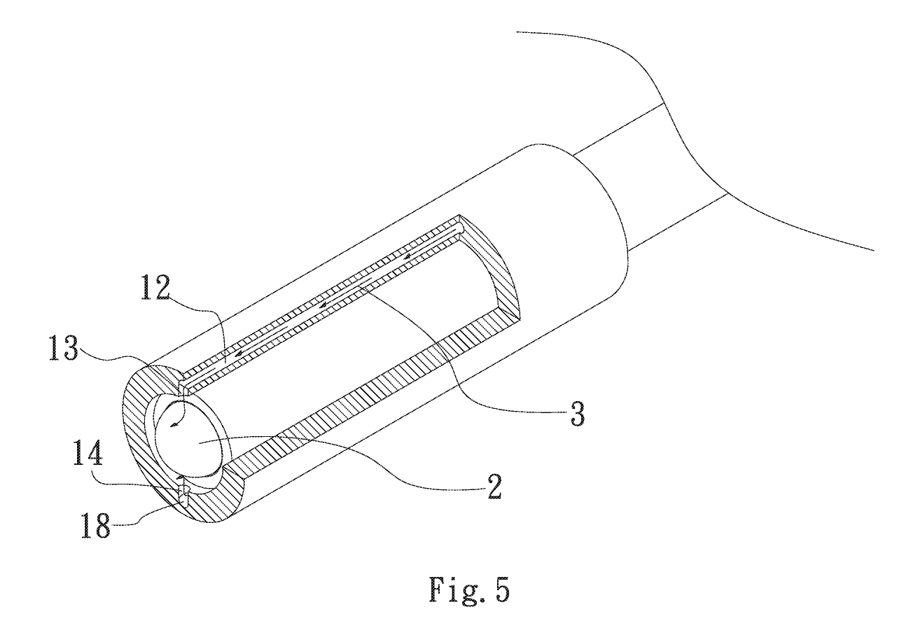

Please now refer to FIG. 5, which is a sectional view of a fourth embodiment of the cleaning structure for endoscope lens of the present invention. The fourth embodiment is partially identical to the first embodiment in structure and thus will not be repeatedly described hereinafter. The fourth embodiment is different from the first embodiment in that the sleeve main body 1 further has a second axial fluid passage 18 and a second opening 14. The second axial fluid passage 18 communicates with the second opening 14. The first axial fluid passage 12 directly communicates with the first opening 13, while the second axial fluid passage 18 directly communicates with the second opening 14. The first and second axial fluid passages 12, 18 are independent from each other without communicating with each other. The first and second openings 13, 14 are radially directed to the lens 2 of the endoscope so that the fluid 3 can directly clean off the dirt on the surface of the lens 2 of the endoscope.

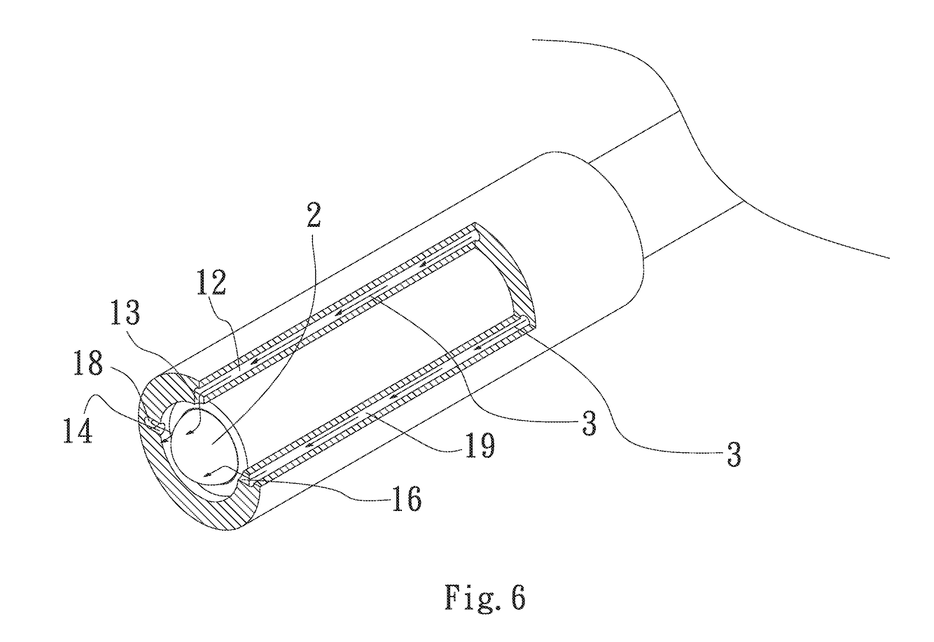

Please now refer to FIG. 6, which is a sectional view of a fifth embodiment of the cleaning structure for endoscope lens of the present invention. The fifth embodiment is partially identical to the first embodiment in structure and thus will not be repeatedly described hereinafter. The fifth embodiment is different from the first embodiment in that the sleeve main body 1 further has a third axial fluid passage 19 and a third opening 16. The third axial fluid passage 19 communicates with the third opening 16. The first axial fluid passage 12 directly communicates with the first opening 13, the second axial fluid passage 18 directly communicates with the second opening 14, while the third axial fluid passage 19 directly communicates with the third opening 16. The first, second and third axial fluid passages 12, 18, 19 are independent from each other without communicating with each other. The first, second and third openings 13, 14, 16 are radially directed to the lens 2 of the endoscope so that the fluid 3 can directly clean off the dirt on the surface of the lens 2 of the endoscope.

It is a primary object of the present invention to provide the cleaning structure for endoscope lens to solve the problem that in working, the lens of the endoscope is often contaminated and covered by dirt to obstruct an operator from doing the inspection work. The cleaning structure for endoscope lens of the present invention is especially applicable to the lens of a medical endoscope. In the case that the lens of the medical endoscope is contaminated and covered by the internal tissue fluid of human body or by the ablated or crushed necrotic tissue, the conventional wiper-like cleaning structure can hardly effectively clean up the lens. The cleaning structure for endoscope lens of the present invention is most different from the conventional wiper-like cleaning structure in that the sleeve main body 1 is fitted around the endoscope. The wall of the sleeve main body 1 is formed with the axial fluid passages. In addition, at least one opening is radially formed at one end of the sleeve main body 1 in adjacency to the lens 2 of the endoscope. In the case that the lens 2 of the endoscope is contaminated, the sleeve main body 1 provides the fluid 3 to clean up the lens 2. The fluid passages of the sleeve main body 1 are directly connected to an external fluid supply pipeline or device, which supplies the fluid to the sleeve main body 1. The sleeve main body 1 of the present invention serves to clean up the surface of the lens 2 of the endoscope. The external fluid supply pipeline or device pertains to prior art and thus will not be further described hereinafter.

When the lens of the endoscope is contaminated by dirt to obstruct an operator from doing the inspection work, the present invention can immediately clean up the lens. Moreover, the present invention is able to continuously eject the fluid to form a shield for preventing the external dirt from getting close to the lens of the endoscope. Accordingly, the lens of the endoscope can keep clean to enhance the working efficiency.

The present invention has been described with the above embodiments thereof and it is understood that many changes and modifications in such as the form or layout pattern or practicing step of the above embodiments can be carried out without departing from the scope and the spirit of the invention that is intended to be limited only by the appended claims.

What is claimed is:

1. A cleaning structure for endoscope lens, comprising:
a sleeve main body having a fitting hole, at least one first axial fluid passage and a first opening, the fitting hole communicating with two ends of the sleeve main body, the first axial fluid passage being axially formed in a wall of the sleeve main body, the first opening being formed at one end of the sleeve main body in communication with the first axial fluid passage.

2. The cleaning structure for endoscope lens as claimed in claim 1, wherein the sleeve main body has a first end and a second end respectively positioned at two ends of the sleeve main body, the first opening being selectively disposed at one of the first and second ends.

3. The cleaning structure for endoscope lens as claimed in claim 1, further comprising a second opening and a circumferential fluid passage, the first axial fluid passage and the second opening being selectively disposed at one of the first and second ends, the first axial fluid passage and the second opening communicating with the circumferential fluid passage.

4. The cleaning structure for endoscope lens as claimed in claim 1, further comprising a second axial fluid passage and a second opening, the second axial fluid passage communicating with the second opening.

5. The cleaning structure for endoscope lens as claimed in claim 4, further comprising a third opening, a fourth opening and a circumferential fluid passage, the third and fourth openings and the circumferential fluid passage communicating with the first and second axial fluid passages and the first and second openings.

6. The cleaning structure for endoscope lens as claimed in claim 1, wherein the sleeve main body is made of a material selected from a group consisting of rubber, silicone, plastic, metal and glass.

7. The cleaning structure for endoscope lens as claimed in claim 1, further comprising a third axial fluid passage and a third opening, the third axial fluid passage communicating with the third opening.

8. The cleaning structure for endoscope lens as claimed in claim 1, wherein the fluid passing through the sleeve main body is a vapor fluid or a liquid fluid.
